(19) United States
(12) Patent Application Publication
(10) Pub. No.: US 2004/0015098 A1
(43) Pub. Date: Jan. 22, 2004

(54) Dynamic ocular visual monitoring system

(76) Inventor: Philippe A. Souvestre, Delta (CA)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 10/200,563

(22) Filed: Jul. 19, 2002

(51) Int. Cl. A61B 13/00

(52) U.S. Cl. 600/558

(57) ABSTRACT

A medical device for capturing eye movement in response to a moving target (20) includes a camera (40) for capturing images of the eye while the eye is focused on the moving target (20). A movement system (86) controls the motion of the target (20). One or more communication interfaces (110) communicate with external devices. The external devices may include a computing device (115) or a recording device (90) for recording the image and target position data. Computing device (115) may include computer executable software components for controlling the movement system (86) and analyzing the images captured by the camera (40), including determining the position of the center of the pupil within each image and plotting that position against the corresponding position of the target (20) when the image was captured.

### FIELD OF THE INVENTION

This system relates to medical devices for diagnosing damage or dysfunction of the human nervous system by monitoring eye movement of a patient in response to a moving target, and then recording and reporting such eye movement correlating to the motion of the target. The report can then assist a physician in diagnosing and treating the patient, and in monitoring the progress and effectiveness of the treatment.

### BACKGROUND OF THE INVENTION

The present invention is an application of the research and development of a new medical and neurophysiological method developed by Applicant, called NeuroKinetics, for diagnosing, treating and monitoring patients with damage to or dysfunction of the central nervous system.

NeuroKinetics analyzes the complex interrelationship between various body parts controlled or regulated by the sensory motor regulators (or controls) within the central nervous system. The patient's ability to control his eye movement is of special interest in the analysis, because it is one of the indicators of central nervous system function and dysfunction.

The NeuroKinetics method utilizes medical technology designed to complete comprehensive functional neurophysiological status analysis to confirm sensory-motor disorders and related Postural Deficiency Syndrome. Postural Deficiency Syndrome, defined in Europe in the early 1980's, is a condition including the combined presence of a high degree of postural pain, visual dysfunction, balance disorders, cognitive disorders, underlying chronic fatigue, and clinical depression. The sensory-motor regulation system responsible for posture and balance is the Fine Postural System. The Fine Postural System includes the musculo-skeletal controls, the ocular-visual system controls, and the auditory and balance control system within the central nervous system. The Fine Postural System functions on a “self-monitored multi-layer” basis. Each “layer” of control sends and receives information from other layers according to pre-set strategies so that each layer may be aware of the positions of other layers. For example, the oculo-motor control layer positions both retinas in relation to the position of the labyrinth and its semi-circular canals, rachis, and lower limbs. Consequently, the position of the pupils can be used to determine the health and functionality of the oculo-motor control layer.

NeuroKinetics relies on the use of specific paradigms relating to the sensory-motor regulation within the central nervous system and brain. Neurophysiological paradigms relate to the body controls within the brain, including physical, structural, cognitive, emotional, intellectual, and behavioral. Updated and new neurophysiological paradigms have been successfully identified and used in several major areas of medicine: 1. Risk prevention and skills optimization in sport medicine, both professional and amateur; 2. Diagnosis and treatment of Chronic Resistant Conditions (i.e., conditions present for 6 months or more that have not responded to reasonable non-NeuroKinetics treatments) as defined in publications such as “Neurophysiopathogenic Aspects of the Postural System” by P. Souvestre, G. Michot; “Posture and Visual Ergometry” by P. V. Bernard and P. Souvestre, and “Postural Consequences of Static and Dynamic Work” by Dr. P. A. Souvestre; 3. Risk prevention and skills optimization in corporate occupational medicine; and 4. Selective evaluation/preparation for operation in extreme/hostile conditions.

The NeuroKinetics method has been successfully applied to diagnosing and treating patients and in monitoring the effects of the treatment. This method has been exceptionally successful in treating patients with ailments such as chronic back-pain, chronic headaches, clinical depression, balance problems, dizziness, chronic irritable bowel syndrome, insomnia, and chronic fatigue. It is not easy to diagnose these types of ailment and select the most effective treatment methods using standard protocols, however, they can be effectively diagnosed using the NeuroKinetics approach. Applicant has found that neurological dysfunction can be corrected while damage to the central nervous system typically cannot. Further, many patients have been misdiagnosed as having a damaged central nervous system, when in fact their symptoms resulted from treatable neurophysiological dysfunction. In the context of work/sport-related acute or chronic resisting complaints, the dysfunction may be the result of frequent exposure to demanding environment constraints or conditions.

As discussed above, eye movement is of special interest in NeuroKinetics, however, eye movement in response to external stimuli is difficult to measure using conventional manual measurement tools. Conventional measuring tools, such as rulers, pupil calipers or protractors are not suitable to measure a fast-moving eye pupil traveling only a minute distance. Those tools are too slow and result in inaccurate measurement. Without objective, rapid, and accurate measurement of eye motion, examination or observation of a patient's eye movement in response to external stimuli involves mostly subjective perception of the physician. Subjective methodologies are difficult to learn and acquire by physicians who have not had specific training and a lengthy apprenticeship with an experienced physician. Subjective aspects of a method may also hinder many physicians who wish to utilize NeuroKinetics methods when treating their patients and in doing so harness the benefits for their patients. Therefore, a need exists for an improved methodology for measuring and/or interpreting eye movement in response to external stimuli.

### SUMMARY OF THE INVENTION

One aspect of the present invention includes a medical device for capturing eye movement in response to a moving target. The medical device includes a control box positioned in front of the patient. A camera is attached to the control box and focused on eyes of the patient. A movement system is also attached to the control box for supporting and moving a target relative to the eyes of the patient. After the patient focuses their eyes on the target, the movement system may begin moving the target. As patient's eyes track the moving target, the camera captures images patient's eyes. In some embodiments, the movement system also communicates the position of the target to other devices or a recorder.

Another aspect of the present invention includes a system for capturing and analyzing eye movement in response to a moving target incorporating the medical device. The system includes a computing device with a processing unit, camera communication interface, movement system communication interface, and a storage medium coupled to the processing unit. The camera communication interface is coupled to both the processing unit and the camera so that images captured by the camera are transmitted to and received by the processing unit. The movement system communication interface is coupled to both the processing unit and the movement system enabling the processing unit to send signals to the movement system to which the movement system responds. The storage medium stores program code implemented by the processing unit including program code for controlling the movement system, determining the position of the target while images are captured; and analyzing eye movement in response to the moving target by analyzing the images captured by the camera.

Another aspect of the present invention includes a method for assisting in diagnosing, treating, and monitoring a patient with a malfunction of the central nervous system utilizing the medical device. The patient is positioned in front of the control box where the target may be positioned in front of the patient's eyes. The camera is positioned to capture a series of images of the patient's eyes. The target is moved along a trajectory relative to the eyes of the patient after the patient has focused their eyes on the target while the camera captures a series of images of the eyes as the target moves. The position of the target is determined at the time the images are captured. The position of a pupil of each eye in each image can then be analyzed. As a further aspect, analyzing the position of the pupil may include determining a coordinate system wherein an origin of the coordinate system appears within each image of the series. The method may also include additional procedures to be performed on each image of the series. Particularly, the method may involve determining the location of the pupil of the eye in the selected image, determining the center of the pupil, and determining the coordinates of the center of the pupil within the coordinate system relative to the origin. The coordinate and position data may then be plotted.

As another aspect, the present invention may include computer executable software components to facilitate analyzing the eye image and target position data. The computer executable components may include an eye movement analysis module. The eye movement analysis module may identify the pupil in each image, determine a coordinate system for the images; determine the coordinates of the center of the pupil, and calculate the distance of the external visual stimulus from the pupil. The computer executable components may also provide program code for displaying a report including the coordinates of the center of the pupil and the distance of the external visual stimulus from the pupil.

As another aspect, the present invention may include analyzing the speed of movement of the pupil while tracking the moving target. To analyze the speed of the movement of the pupil, the method selects successive pairs of images captured while the target was moving. The method then determines the coordinates of the center of the pupil in both images. Next, the method determines the distance between the centers. The method next determines the time interval that elapsed between capturing the images. The speed of movement can then be calculated as a function of the distance between the centers and the time interval that elapsed between the images.

### BRIEF DESCRIPTION OF THE DRAWINGS

FIG. 1 is an overall isometric view of a first embodiment of the present invention.

FIG. 2 is a top view of a control box assembly in the first embodiment.

FIG. 3 is a side view of a control box assembly in the first embodiment.

FIG. 4 is a front view of a control box assembly in the first embodiment.

FIG. 5 is a background screen of the first embodiment.

FIGS. 6A and 6B are examples of an eye movement plot obtained by an embodiment of the present invention.

FIGS. 7A-10B are examples of eye movement plots, showing a patient's progress during treatment.

FIG. 11 is a block diagram depicting a system incorporating medical device 10.

FIG. 12 is a block diagram depicting an illustrative architecture of a computing device capable of implementing an eye movement analysis module formed in accordance with the present invention;

FIG. 13 is a flow diagram depicting a method of data collection using medical device.

FIG. 14 is a flow diagram illustrative of the eye movement analysis module implemented by the computing device in accordance with the present invention;

FIG. 15 is a flow diagram illustrative of the determine focused pupil coordinates component of the eye movement analysis module.

FIG. 16 is a flow diagram illustrative of the determine speed of eye motion component of the eye movement analysis module.

### DETAILED DESCRIPTION OF THE PREFERRED EMBODIMENT

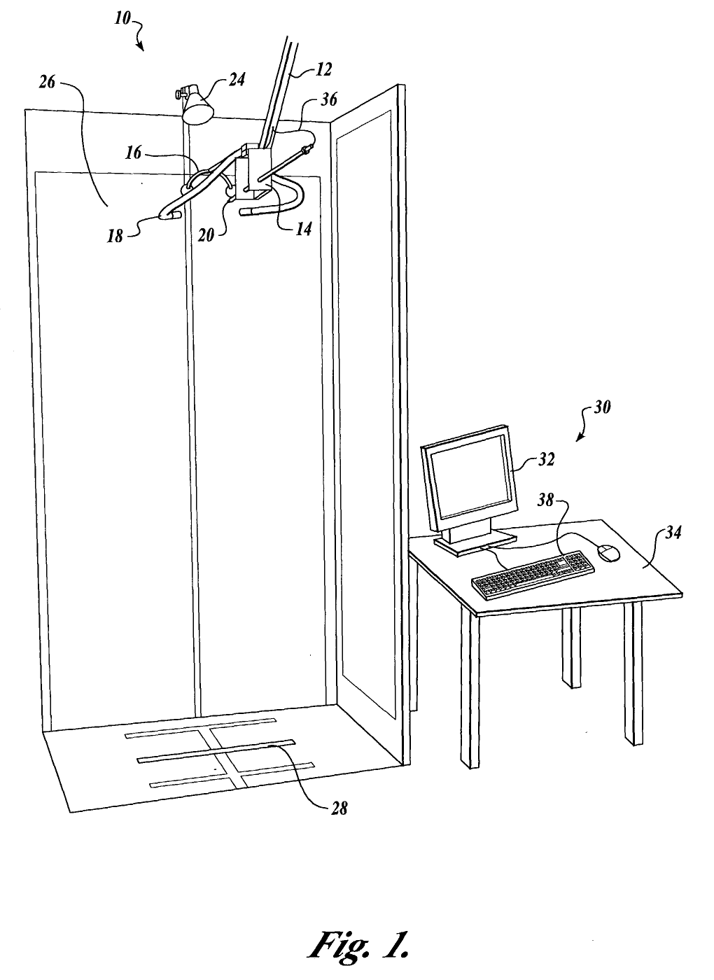

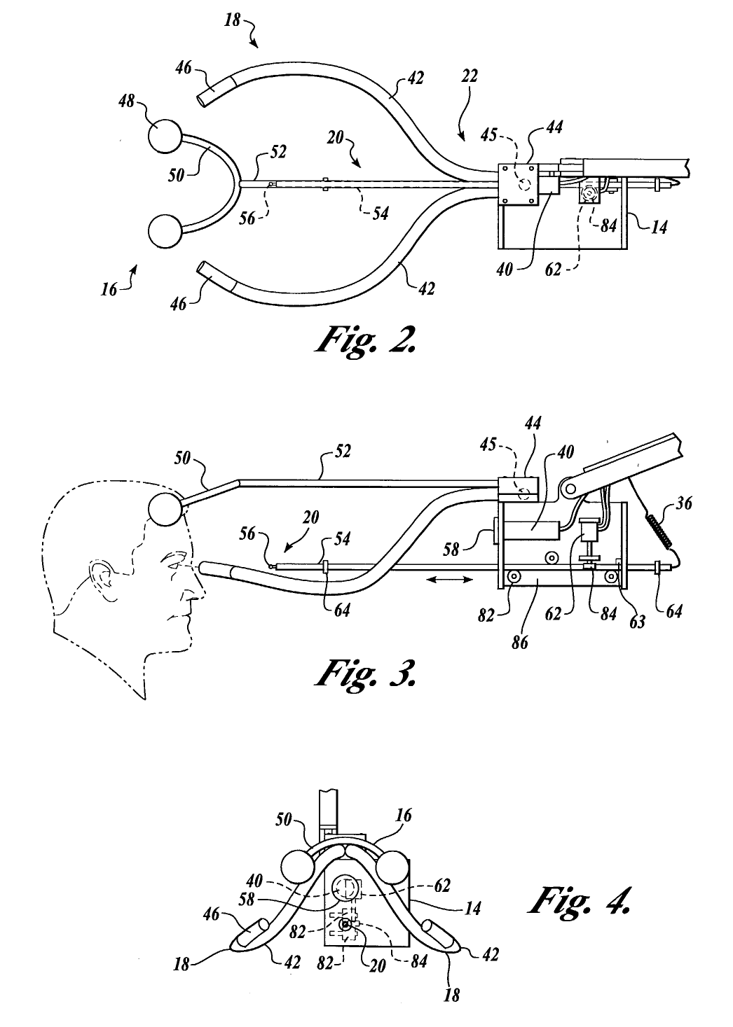

FIGS. 1-4 provide a general view of one embodiment of the present invention, a medical device 10, or a Dynamic Ocular Visual Monitoring System. Medical device 10 includes a moveable target 20 supported and controlled by a movement system 86. The target 20 is positioned in front of a patient so that the target may be viewed by the patient. As the movement system 86 moves the target 20, medical device 10 collects position data concerning the position of the target 20 and camera 40 captures images of the patient's eyes.

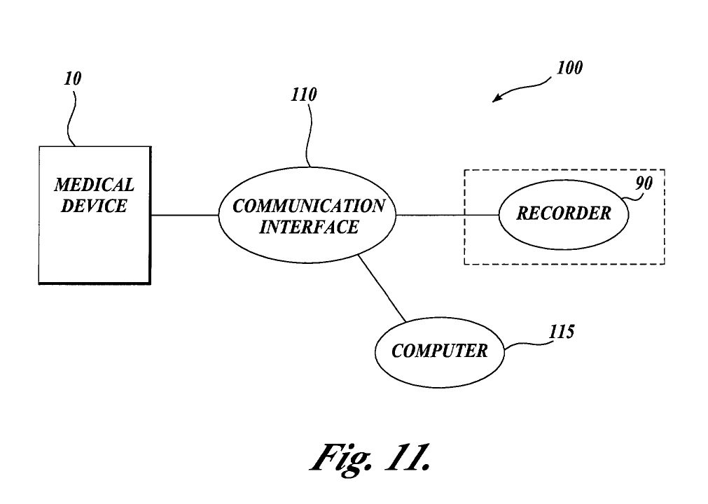

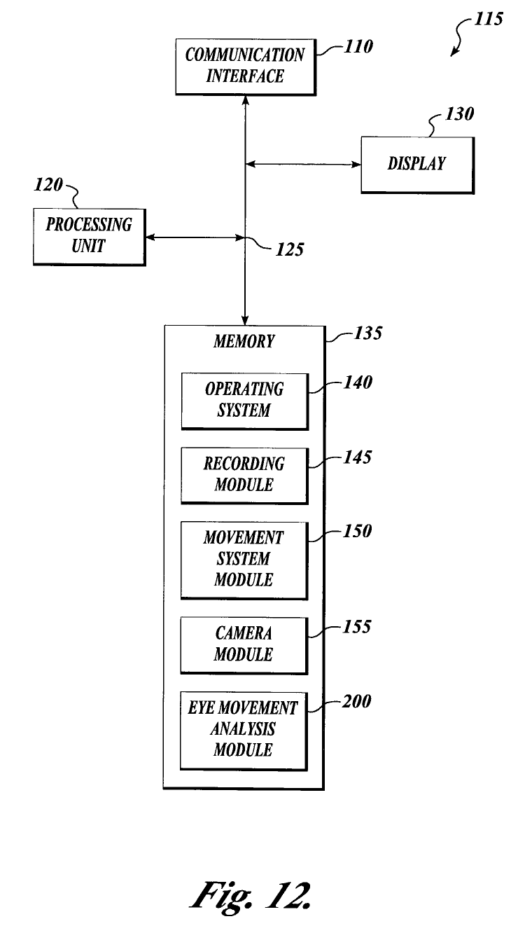

In one embodiment depicted in FIG. 11, the medical device 10 uses a communication interface 110 to communicate the data collected to other devices, such as computer 115 and/or recorder 90 forming system 100. In another embodiment, the data collected may be analyzed using software 200 (depicted in FIGS. 12-16) and executed on computer 115. In another aspect of the present invention, data collected by medical device 10 may be analyzed and plotted to produce plots like the ones shown in FIGS. 6A-10B.

The medical device 10 will now be discussed in greater detail.

### Medical Device 10

A control box 14, is the “center” of the medical device 10. Most of the data collection instruments are housed either inside the control box 14 or are attached to it. As shown in FIG. 1, a headrest 16, eye illuminators 18, and a target 20 are carried by the control box. The control box 14 is in turn carried by one end of an adjustable, articulatable support arm 12. At the other end, the support arm 12 may be hung from the ceiling, a mobile stand, or a sidewall of an examination room. With the support arm 12, the position of control box 14 can be adjusted to suit different patients and other examination needs.

A grid pattern 28 is located on the examination room floor. During an examination the patient stands at a location as indicated by the grid pattern 28. Optionally, a background screen 26 may be positioned a distance away from the control box 14. The background screen 26 may have multiple panels that may be arranged to form a semi-enclosed space for patient examination. A light 24 may be located above the background screen 26 to illuminate the general examination area. Preferably, the background screen 26 is spaced from the grid pattern 28 and does not encroach upon it. In this manner, the patient may be surrounded by the background screen 26 at the sides and back while facing the control box 14. The surfaces of the background screen 26, the grid pattern 28, and most of the equipment in the examination room may be made of or coated with anti-reflection material to reduce sound and reflective light in the examination area.

With respect to the description of the present invention, the direction between the patient and the camera in the horizontal plane defines Z direction. The direction perpendicular to the Z direction in the horizontal plane is the X direction. The vertical direction is the Y direction.

FIGS. 2, 3, and 4 illustrate in more detail the control box 14 of the present invention shown in FIG. 1. FIG. 2 is a top schematic view of the control box 14 and the surrounding instruments. The control box may be generally cubic in shape, any suitable rectilinear shape, or any other suitable shape defining a sufficient volume, such as a sphere, tetrahedron, octahedron, dodecahedron, etc.

An eye illuminator assembly 22 may be attached to the control box 14. The eye illuminator assembly 22 may include two eye illuminators 18, one located on each side of the control box 14 and projecting forwardly from the control box 14. Eye illuminator assembly 22 may also include a light box 44 that is attached to the proximal ends of the eye illuminators. In one embodiment, the light box 44 may be located on the top forward portion of the control box 14. Each eye illuminator 18 includes an arm 42 that may be constructed from a flexible, gooseneck-type conduit. At distal end of each eye illuminator arm 42 is an opening, forming an eye illuminator head 46. A fiber optic bundle extends through the arm 42 to transmit light from a light bulb 45 that is sealed inside the light box 44, to the distal end of the eye illuminator arm 42, at which the eye illuminator head 46 is located. The intensity of light from the light bulb 45, or any other light emitting devise used, may be adjustable. The light box 44 is sealed so light is only transmitted from the eye illuminator heads 46. The light intensity at the eye illuminator heads 46 may be controlled to be from 0-90% of the maximum intensity of the light bulb 45. The eye illuminator heads 46 may be covered by optical filters during use to enhance the contrast of eye images.

A headrest 16 is located between the two eye illuminators 18. Any suitable headrest configuration known in the medical arts may be used such as the headrest 16 depicted in FIGS. 2-4. The headrest 16 may include two laterally spaced-apart headrest tips 48 located at the distal ends of a U-shaped headrest arc 50. A straight, longitudinal arm 52 extends from the center of the arc 50 to within the control box 14. In an alternate embodiment, a generally U-shaped headrest pad may be connected to the longitudinal arm 52. In this manner, the U-shaped headrest pad will rest directly upon the head of the patient. The U-shaped headrest pad may include disposable or re-useable cover to facilitate cleaning the U-shaped headrest pad. In yet another embodiment, a headrest section or sections may be formed into the headrest arm 52 so that the free end of the headrest arm 52 contains bulbous or U-shaped sections as required to form a headrest. Either the headrest arm 52, or the U-shaped arc 50, may be bent slightly in the downward direction near the headrest tips 48, to provide a clearer view of the patient's eyes to a camera 40 located inside the control box 14. While in the embodiment depicted in FIGS. 1-4 the headrest 16 does not immobilize the head of the patient, it is desirable to maintain the head in a fixed position relative to the camera 40. In alternate embodiments, headrest structures that control or limit movement of the patient's head may be used.

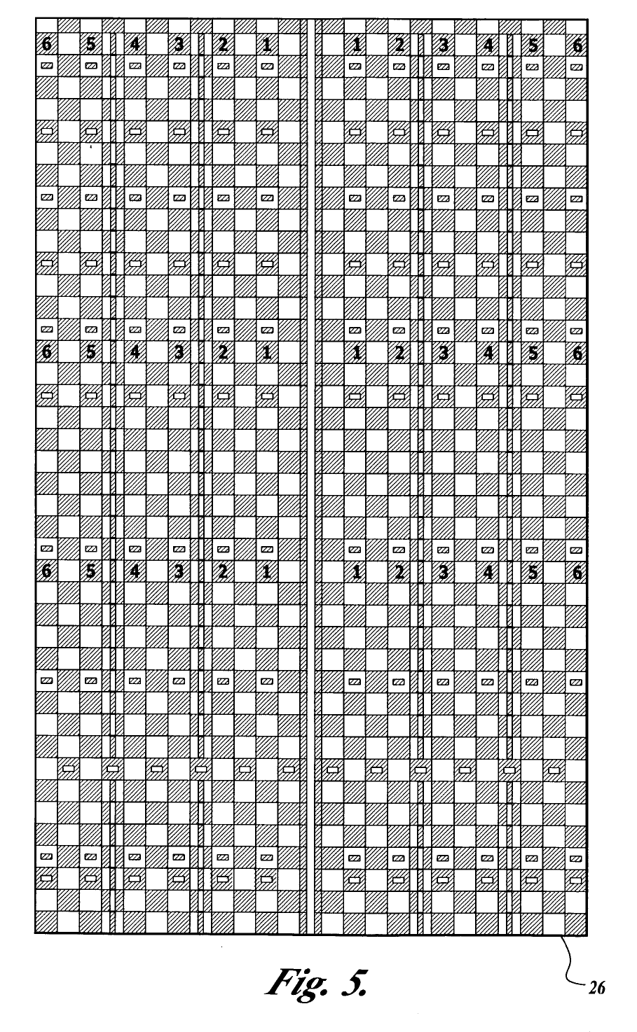

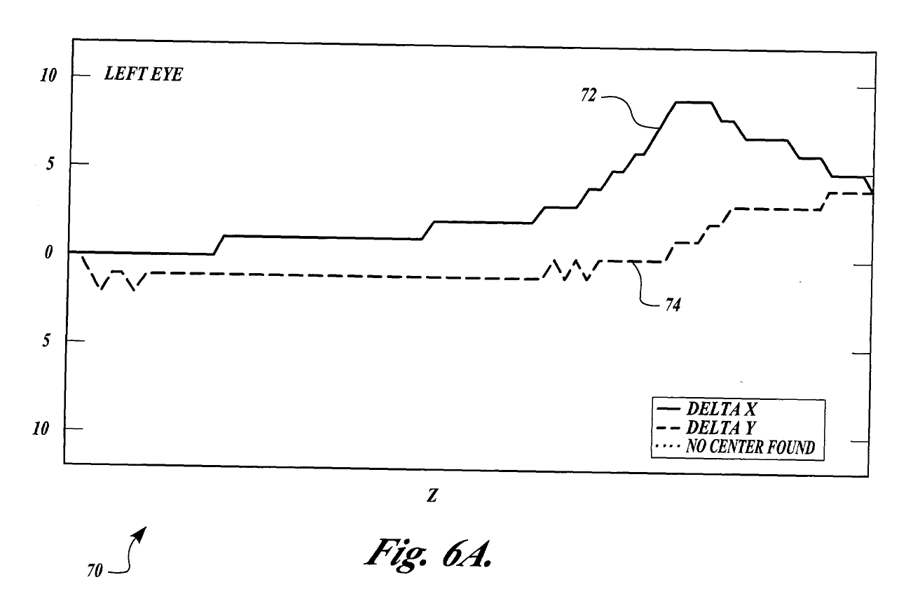

FIG. 3 is a side view of the control box 14 and the related components of the present invention. The camera 40 is housed within the control box 14. The camera 40 may be located at about the middle of the control box 14 and is held in place by brackets or other suitable supporting structure. A camera window 58 may be located at the front of the control box, near the center thereof, through which the camera 40 can capture eye images of a patient. The light box 44, mentioned above, may be located at the top of the control box 14 to provide light for the eye illuminators 18. A light bulb 45 may be sealed inside the light box 44. Also, as shown in FIG. 3, one end of the headrest arm 52 is supported by an upper portion of the control box 14.

Below the camera 40 is a spherical target structure 20 mounted on the distal end of an extension-retraction rod 54. The target 20 may include an LED light 56. A movement system 86 may support the target structure 20 and move the target structure 20 relative to the eyes. The movement system 86 may also control the position and speed of movement of the target structure 20. In one embodiment, the movement system 86 includes idle wheels 82 that support and engage rod 54 and also bear against the underside of the rod 54. The movement system 86 may also include a driving wheel 84 that contacts the upper surface rod 54 as is driven by a motor 62 to extend and retract the rod 54 relative to the box 14. In one embodiment, the rod 54 is hollow so that electrical wires may extend through the rod 54 to supply electric power to an LED light 56 located at the distal end of the rod 54. In one embodiment, the movement system 86 includes safety stops 64 that be located adjacent each end of the rod 54. The stops 64 bear against portions of the box 14 to limit the extension and retraction of the rod 54 relative to the box 14. The stops 64 prevent the LED light 56 from accidentally contacting the face of the patient when traveling forwardly and also prevent the rod from retracting too far into the box. Other types of interlocking or safety devices may be employed instead of stops 64. The individual power cables and signal cables for the LED light 56, the motor 62 or the camera 40, are bundled together to form the data and power cable harness 36. The movement system 86 may also output information concerning the position of the target 20 in a recognizable format (described in more detail below).

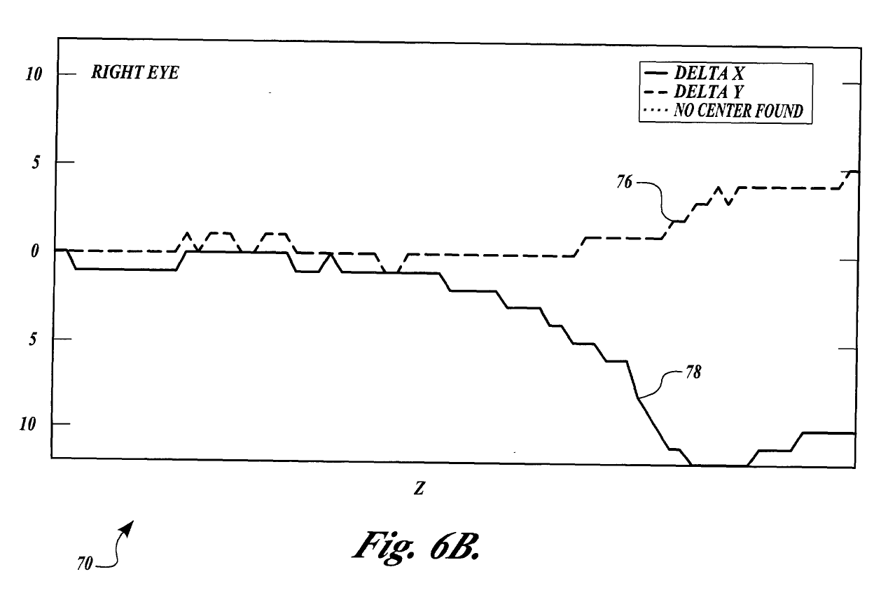

FIG. 4 is a front view of the control box 14 and the related components of the present invention. The two eye illuminators 18 and the headrest 16 are located at the upper portion of the box 14. The camera window 58 and the camera 40 are located generally centrally of the box. The target 20 and movement system 86 including actuating motor 62, driving wheel 84 and idle wheels 82, may be positioned at a lower portion of the control box 14.

The FIGS. 1-4 show one embodiment of the present invention. There are many other ways to implement the various functions of the present invention. For example, the target 20 need not include an LED light. The target 20 can be lighted by other means. Further, any small object movable toward and away from a patient's eyes can be used as a target upon which the patient may focus their eyes. The current invention can be used to measure the focus of the patient's eyes on either a point light source in a dark space or a dark object in a lighted space. Either test modality may be used.

The LED light 56 need not be mounted on the end of a straight hollow rod, such as rod 54. In an alternate embodiment, the movement system 86 may include a line, cable, or an endless belt moving linearly towards and/or away from the patient's eyes to which the LED light 56 may be attached. In addition, the movement system 86 could include more elaborate motion mechanisms to generate two-dimensional motion, i.e. moving towards the patient's eyes and simultaneously moving sideways, or even three-dimensional motion, i.e. moving towards the patient's eyes and simultaneously moving up and down and sideways. A target moving in a 2- or 3-dimensional trajectory may be useful in pinpointing patient's problem in a particular area of the central nervous system. The embodiment of target 20 and movement system 86, shown in FIGS. 1-4, has the advantage of being quite simple while still having the capability to generate pseudo 2- or 3-dimensional movement, as discussed below. In one embodiment, the target light 56 has a range of movement of about 400 mm. The speed of movement of the target 20 can vary from 0 mm/sec to a top speed of about 100 mm/sec.

The camera 40 need not to be any particular type, however, the camera 40 must capture an adequate number of frames within a short period of time and at an adequate resolution. For example, the camera may take 10-60 frames (or pictures) per second for a few seconds or several minutes. The camera may have a resolution of, for example, 50-200 pixels or dots per eye, or several hundred pixels for both eyes. An adequate resolution may be able to detect eye movements as small as from about 0.5 to 0.1 mm.

The camera 40 captures image data that may be analyzed in real time as the frames of the image data are collected or recorded and stored for analysis at another time. The type of camera 40 selected for use within a particular embodiment will depend upon when the user desires to analyze the data. Consequently, either a still camera or video camera may be used. If recordation of the images is desired, the camera 40 may be connected to a communication interface 110 that will allow the camera 40 to communicate with external recording devices such as recorder 90 or computer 115 (see FIG. 11). Alternatively, image data may be recorded using recording media that is incorporated into the camera 40. Recording media may include optical film, electronic media, or others. For an optical film based camera, the photographic film serves as the data recorder. The images of eyes and the target positions may be recorded and/or coded onto the film. Images captured on optical film may be analyzed manually or digitized (such as by scanning the negatives or printed images) so that software (such as the eye movement analysis module 200) may be used to perform the analysis. If an electronic media-based camera is used, either analog or digital cameras may be employed. The images and target positions may be recorded on various media, such as videotapes, removable magnetic discs, CDs, removable memory chips, computer hard-drives etc. An electronic digital camera with computer readable recording media has the advantage that the digital format employed makes it easier to integrate the data collection process with the data analysis process, especially when a computer is used in data analysis.

If a separate recorder 90 is used a communication interface 110 may be used to connect a recorder 90 to the camera 40. Separate recorders may include video cassette recorders, digital video devices, magnetic disc writers, CD writers, and the memory 135 of computer 115. In one embodiment, the computer 115 includes a recording software module 145 for capturing and storing images captured by camera 40.

If real time data analysis is desired, the communication interface 110 may be used to connect a computer 115 directly to the camera 40 so that images can be analyzed as they are connected. Images analyzed in this manner may be optionally recorded in the memory 135 of computer 115 or using the communication interface 110 recorded by a separate recorder 90. Alternatively, the communication interface may be used to connect the computer 115 to the recorder 90 so that the computer can analyze images as they are recorded.

Using a digital camera may facilitate incorporating the memory 135 of computer 115 across the communication interface 110 and using memory 135 as the recorder 90. Electronic digital format also allows for convenient data transmission between different physical locations through telecommunication channels, so physicians at different physical locations can work together in treating patients. Data transmission can occur by physically transferring the media on which the image data is captured or using a communication interface 110 to connect computer 115 with telecommunication channels such as local area networks, dedicated telephone lines, data transmission lines, or the Internet.

One or more emergency handheld controllers or start/stop switches may also be incorporated in the medical device 10. During the exam, the patient may hold an emergency switch, whereby the patient has the ability to stop the examination at any time.

FIG. 5 shows an optional background screen 26 that may be used with one embodiment of the present invention. The background screen 26 may provide a reference as to the position of the target 20, the eye illuminators 18, and the patient. The background screen 26 may also provide a reference as to the intensity of the illuminating light.

### System 100

The system 100 illustrates one embodiment of a system incorporating the medical device 10. The system 100 may include medical device 10 connected to a communication interface 110 that is coupled to computer 115. Optionally, a recorder 90 can be connected to a communication interface 110 that is connected to the medical device 10. In this manner, the medical device 10 can communicate images and data to the recorder 90 for recordation and storage.

Returning to FIG. 1, a physician's workstation 30 may be located adjacent to, near, or outside the examination area. A desk 34, a computer monitor 32, and a keyboard 38 principally comprise the physician's workstation 30. Optionally, computer 115 may be located at or near the physician's workstation 30. The physician's workstation 30 may also include a plotter or printer used for printing plots or reports.

While a single computer 115 and communication interface 110 are depicted in FIGS. 11 and 12, it is apparent to one of ordinary skill in the art that the operations disclosed in this application can be divided among several computers. Furthermore, each device comprising system 100 may require a separate communication interface to communicate with one or more devices in the system 100. Therefore, communication interface 110 is representative of the software and hardware components necessary to effect communication between the various devices of system 100.

As shown in FIG. 12, the computing device 115 includes a processing unit 120, a display 130, and a memory 135. The memory 135 generally includes a random access memory (“RAM”), a read-only memory (“ROM”), and a permanent mass storage device, such as a hard disk drive, tape driver, optical drive, floppy disk drive, CD-ROM, DVD-ROM, or removable storage drive. The memory 135 stores an operating system 140 for controlling the operation of the computing device 115. The memory may also store a recording module 145, movement system module 150, camera module 155, and eye movement analysis module 200 of the present invention. It will be appreciated that the operating system 140 and aforementioned modules may be stored on a computer-readable medium and loaded into memory 135 of the computing device 115 using a drive mechanism associated with the computer-readable medium, such as a floppy, CD-ROM, DVD-ROM drive, or network interface. The memory 135 and display 130 are connected to the processor 120 via a bus 125. The display 130 may include computer monitor 32. Other peripherals may also be connected to the processor in a similar manner.

As shown in FIG. 12, in one embodiment of the invention, the computing device 115 may include a communication interface 110 coupled to the processor 120 for establishing a communication link with external devices, computers, and computing networks. The communication interface 110 may include a modem for connecting to an Internet service provider through a Point-to-Point Protocol (“PPP”) connection or a Serial Line Internet Protocol (“SLIP”) connection as known to those skilled in the art. The modem may utilize a telephone link, cable link, wireless link, Digital Subscriber Line or other types of communication links known in the art. In another embodiment, the communication interface 110 may include a network interface for connecting directly to a LAN or a WAN, or for connecting remotely to a LAN or WAN. Those of ordinary skill in the art will appreciate that the communication interface includes the necessary circuitry for such a connection, and is also constructed for use with various communication protocols, such as the TCP/IP protocol, the Internet Inter ORB Protocol (“IIOP”), and the like. The communication interface may utilize the communication protocol of the particular network configuration of the LAN or WAN it is connecting to, and a particular type of coupling medium. Additionally, the communication interface 110 may include a control rack. The control rack may include analog to digital (A/D) and digital to analog (D/A) converters or control boards, to convert analog or digital signals and communicate between the data acquisition instruments and the computer 115.

The communication interface 110 may also include a data and a power harness 36 connecting the control box 14 to the computer 115 at the physician's workstation 30. Commands or instructions from a physician can be transmitted from the computer 115 via the data and power harness 36 to the control box 14, and the data collected by control box 14 may be transmitted by harness 36 to computer 115, and also may be displayed on the monitor 32.

In one embodiment, software installed on the computer 115 may be used as a controller to control the camera 40 and/or the movement system 86. In one embodiment, the memory 135 of the computer 115 includes a camera module 155 that may be used as a camera controller to control the camera 40. The camera module 155 may allow the user to control the zoom, focus, frame rate (shutter speed), filters, color balance, light settings, and other settings supported by the camera 40. Additionally, the camera module 155 could be used to turn the power to the camera 40 on and off.

In one embodiment, the memory 135 of the computer 115 includes a movement system module 150 that may be used as a movement system controller to control and position the movement system 86 rendering the target 20 in the desired position. During data collection, the movement system module 150 may also control the movement of the movement system 86 including the direction and speed of movement. In another embodiment, the movement system module 150 records the position of the target 20 for each frame of the image data collected.

In one embodiment, the position of the target 20 can be calculated from the control information or signal sent to the drive mechanism such as the motor 62 by the movement system controller. Depending on the type of the motor used, the distance traveled by the target 20 can be calculated from the number of revolutions or the number of steps the motor 62 rotates. Additionally, the position of the target can be calculated from the travel distance of the target, rate of travel, and either the start or end time of travel.

In another embodiment, the movement system module 150 receives position information from the movement system 86 for recordation. One or more sensors 63 may be used to determine the position information. For example, the motor 62 may include an internal sensor, which counts the number of revolutions or steps the motor has turned. To ensure that the information from the motor 62 is accurate, other sensors also may be used to measure the distance the target 20 travels during the examination. For example, a rotational sensor can be used in conjunction with one of the idle wheels 82. Of course, for this measurement of distance to be accurate, the rod must be held snuggly between the idler wheels 82 with no relative slippage therebetween. Similarly, there should be no relative slippage between the driving wheel 84 and the rod 54. Any sensors used to collect target position data must be connected to a communication interface so that information collected can be communicated to either computer 115 or recorder 90.

The position of the target can be recorded periodically or calculated from system variables. For example, the system may record the position of the target as each image is taken. Alternatively, the system may record the trajectory or path that the target will take, the speed at which the target is moving, and the start time of the initiation of motion. From these values, the position of the target may be calculated for any period of time. In yet another embodiment, the target may travel a known distance and the images may be captured at a constant rate. In this manner, the position of the target can be calculated by dividing the distance of travel by the number of images captured. If the position is periodically recorded, it may be recorded as each image is captured or at predetermined time intervals independent of image capture. If the latter method is used, the recorded target positions may be used to interpolate the target positions at the time a particular image was captured.

### Data Collection

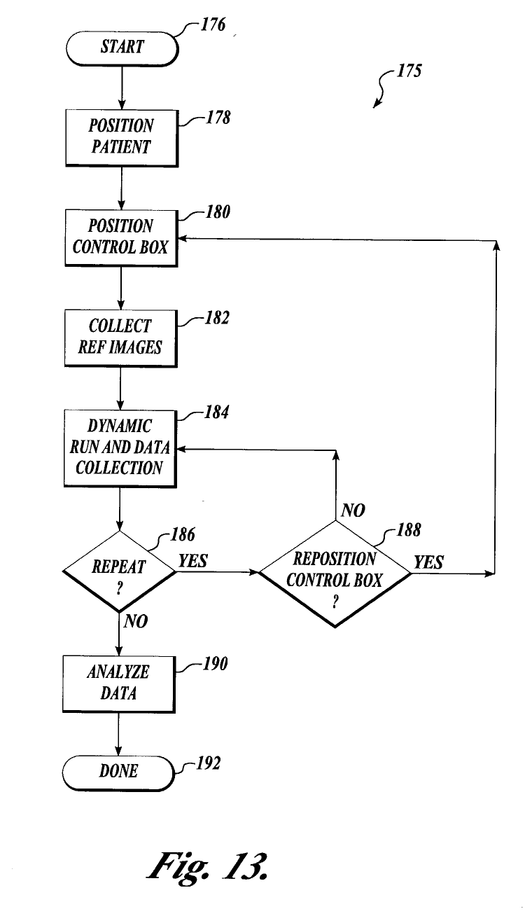

FIG. 13 depicts one embodiment of the general procedures 175 that may be followed during data collection. Data collection starts in block 178 by positioning the patient on the floor grid pattern 28. During examination, a patient is asked to stand, sit, or, if necessary, lay down at a location indicated by the floor grid pattern 28 (depicted in FIG. 1). The patient stands facing the control box 14 and the camera 40. If the optional background screen 26 is used, the patient stands between the control box 14 and the background screen 26.

Next, in block 180 the control box 14 is positioned relative to the patient. The height and position of control box 14 is adjusted by moving the support arm 12 to match the height of the patient. The headrest 16 is pressed against the patient's forehead to fix the distance between the patient's eye and the camera 40. The patient's eyes are illuminated by the eye illuminators 18. Depending on the eye color and size, the positions and intensities of the eye illuminators 18 may be adjusted by either adjusting a light intensity (dimmer) switch connected to the light box 44 or by repositioning the eye illuminator arms 42 and eye illuminator heads 46 to achieve an optimal contrast between the pupils and the rest of the eyes. Optical filters or patterns may cover the eye illuminator heads 46 to change the color of the illuminating light. Sufficient contrast between the pupils and the rest of the eyes aids in identifying the pupils, differentiating whether the eyes are focused or not, and locating the centers of the pupils for more accurate measurement of the motion of the eyes.

Positioning the control box 14 includes ensuring that the target 20 will follow the desired path or trajectory relative to the patient's eyes. Usually, during most examinations, the target 20 moves in the Z direction at a location centered between the two eyes, i.e. the target 20 moves in a linear trajectory defined by the function x=0 and y=0. Sometimes, it may be necessary to position the target 20 off center relative to the eyes, but still move the target purely in the Z direction, i.e. the target 20 moves in a trajectory defined by the function x=a and y=b, where at least one of a or b is not zero. In this manner, the eyes may be caused to move in both the X and Y directions and perhaps causing each eye to move at a different rate.

Alternatively, the target may be moved in a tilted trajectory, such as defined by function x=0, y=tan(β)*z, where β is the angle between the trajectory and the Z direction. Following this trajectory, the target is moving in the Y direction as well as in the Z direction. If the target follows a trajectory defined by the function x=tan(α)*z and y=tan(β)*z, where α is the angle between the projection line of the trajectory on the X-Z plane and the Z direction, β is the angle between the projection line of the trajectory on the Y-Z plane and the Z direction, then the target is moving in a pseudo 3-D pathway. The different target trajectories can be achieved by adjusting the control box 14 to move laterally, up or down, left or right, or angularly.

While the figures depict that the target follows a generally linear trajectory, it can be appreciated by one of ordinary skill that a curved or circular trajectory is also within the scope of the invention. Such a trajectory could be effected by bending or arcing rod 54 and rotating rod 54 in addition to moving extending and retracting the rod 54. In this manner, the LED target 56 will travel along an arc instead of a straight linear path.

Then, in block 182 reference images are captured. Because even a patient with severe dysfunction of or damage to the central nervous system can usually focus their eyes on a stationary target, located a relatively long distance away, even for some length of time, reference images are taken of the focused eyes with the target in a stationary position. Those reference images can be used as baseline images for identifying the focused eyes, identifying the centers of pupils, and determining the distance between the two eyes or the distance between the eyes and the inner corners of the eyes. If software, such as the eye movement analysis module 200, is used to perform the data analysis, these reference images can also help “fine-tune” or calibrate the software to better adapt to the images of the particular patient. Reference images may also be captured of the unfocused eye. To capture these reference images, the patient may be asked to relax the eyes so that the eyes become unfocused. Reference images of the unfocused eyes may be captured by the camera 40 and stored for later use. As a general rule, focused eyes have smaller, more definite pupils than unfocused eyes that have larger, more dilated pupils.

Once the patient and the device 10 are ready and reference images have been captured, a dynamic run may begin in block 184. Returning to FIG. 3, a dynamic run may begin with the lighted LED target 56 in a position closer to the control box 14 so that it may move in the Z direction toward the patient's eyes. The patient is then asked to focus their eyes on the lighted LED target 56. Next, the LED target 56 may be moved towards the patient's eyes. The LED target 56 may be moved toward the patient's eyes actuating motor 62. Actuation of motor 62 will cause the driving wheel 84 to turn against the rod 54 and move the rod 54 in the desired direction. The motor 62 may be actuated by the computer 115 located at the physician's workstation 30. A movement system module 150 (shown in FIG. 12) installed on the computer 115 may provide the logical and interface components necessary for the user to control the movement system 86.

By focusing on the LED target 56, the patient's eye will move in response to the movement of the LED target 56. Any movement of the eyes during the examination is captured by the camera 40. The eye movement image data captured by the camera 40 and the position and speed of the target 20 data may be transmitted through the harness 36 to the computer 115. As shown in FIG. 12, computer 115 may include a recording module 145 that may be used to provide the necessary logic and interface components to record the transmitted data. Additionally, an eye movement module 200 (discussed below) may be used to process and analyze the data. In an alternate, embodiment, the information may be processed or analyzed in a different location through the use of telemedical technology. The length of the dynamic run may be determined by a predetermined time interval or after the target 20 has traversed a desired distance. As one non-limiting example, 10 seconds of dynamic data may be collected.

Because several runs may be performed, the user decides whether to perform another dynamic run in decision block 186. The user may decide to change to the position of the control box 14 in decision block 188 and return to block 180. After repositioning the control box 14, the user may collect new reference images if necessary. When an adequate number of dynamic images of the eyes have been captured, data analysis of the images captured can be performed in block 190. During data analysis, a physician may compare the images of eyes taken during movement stimulated by a moving target with the reference images of the focused and/or unfocused eyes. A computer analysis report generated by the eye movement analysis module 200 (discussed below) may also be used for interpretation. The physician can objectively: determine whether each eye is focused or not; locate the center of the pupil; determine the distance between the center of the pupil and the inner corner of that eye; and view the motion of the eye between sequential images. From the data for the position of the eye, the physician can correlate the eye movement relative to the movement of the lighted target. The relationship between the eye and the target position can be plotted; for example, see the plots shown in FIGS. 6A-10B. This objective data can help a physician diagnose, evaluate the condition of a patient, and monitor the patient's reaction or progress in response to treatment.

### Plots

FIGS. 6A and 6B show an example of a plot 70 generated by use of the present invention. Plot 70 comprises two sections, one for the left eye depicted in FIG. 6A, the other for the right eye depicted in FIG. 6B. The horizontal axis is the target time and distance in Z direction, i.e. the distance between the target and the eye. In this non-limiting example, the plot 70 was made from image data collected for a duration of 10 seconds. The vertical axis is the distance an eye has moved in X or Y direction. There are four lines in plot 70. Referring to FIG. 6A, line 72 represents the horizontal or X direction movement of the left eye, and line 74 represents the vertical or Y direction movement of the same eye. Similarly, as best viewed in FIG. 6B, line 76 indicates the vertical or Y direction movement of the right eye, and line 78 indicates the horizontal or X direction movement of the right eye.

A normal person can focus both eyes at a point target. When the target moves closer and closer towards a point between the two eyes, the left eye will move to the right and the right eye move to the left, as shown in FIGS. 6A and 6B. Under normal conditions, the eye will track the target and change position in each frame of the image data to correspond the position of the target. If the eye is able to follow smoothly the target and not loose focus, plotting the position of the pupil of the eye against the position of the target will generate a plot of data points. A regression analysis of these data points may result in a generally sloped line. In many cases, however, the eye will not be able to track the target during the entire dynamic run. When the eye becomes unfocused, the position of the pupil will not correspond to the position of the target. Under these circumstances, the data point will fall outside the sloped line and appear as a spike or dip in the data plot. Because the line is sloped, it may be difficult to determine visually which data points represent unfocused eye movement.

For ease of interpretation, it may be desirable to center vertically the plot about a reference line, i.e., remove the slope of the line. Any method known in the art may be used to vertically center the plot about the reference line. Suitable methods may include using a constant, a look-up table, or calculating the slope using linear regression. As one non-limiting example, the plots in FIGS. 6A-10B have been centered vertically about the horizontal line passing through the zero-point on the vertical axis. In this manner, the determination of whether the eye is focused is simplified. Data points vertically outside the reference line represent instances during which the eye was unfocused. As can be viewed in right most portions of FIGS. 6A and 6B, when the target moves to a certain distance to the eyes, the eyes may lose either their focus or ability to track the target. At this portion of the plots, the target is nearest the eyes of the patient and may be too close for the focal length of the eyes.

If the target is moving in the same horizontal plane as the eyes, then the eyes may not move upward and downward. But if the target moves in a plane that is above the eyes, then the eyes will move upward somewhat as the target moves closer to the patient, as shown in FIGS. 6A and 6B, line 74 and line 76. To pinpoint a patient's problem, the target may need to move in a different plane or at an angle relative to the horizontal direction.

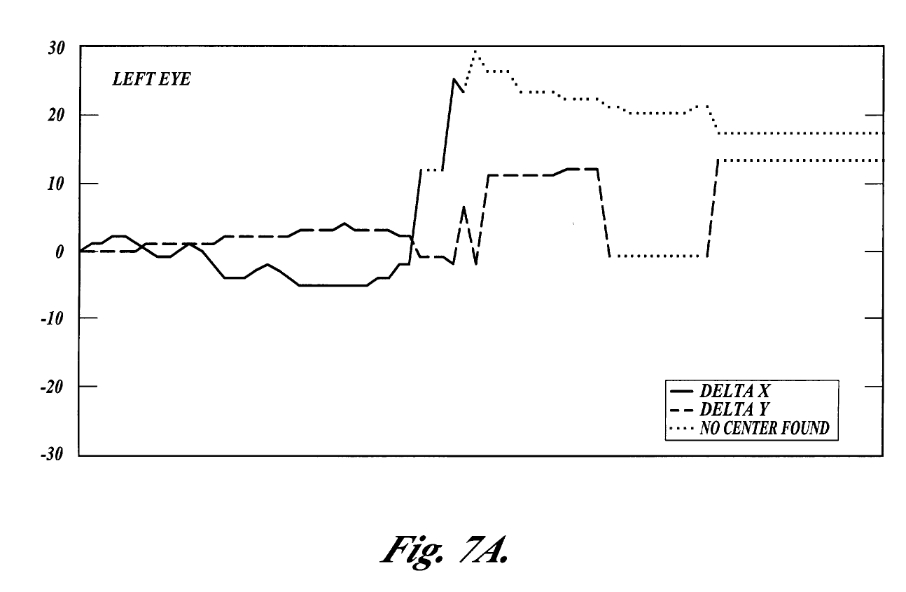

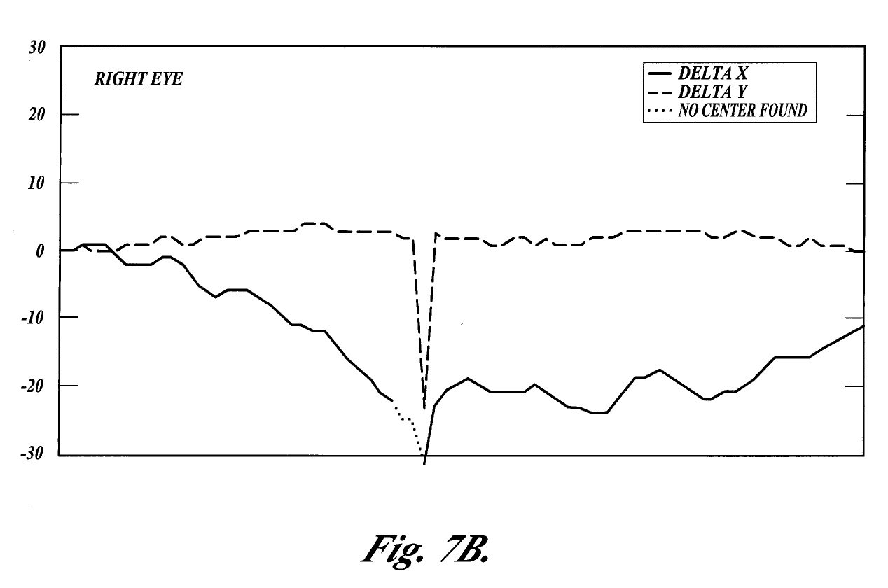

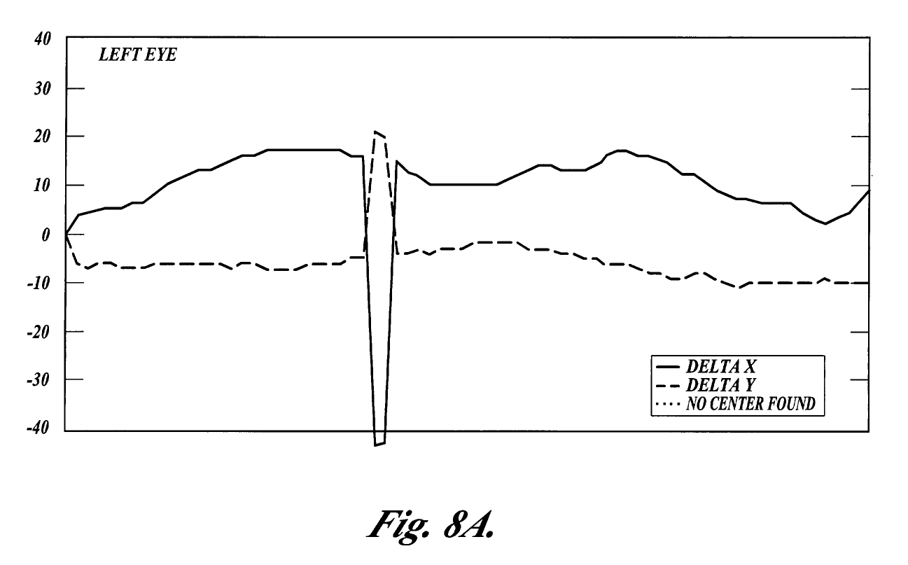

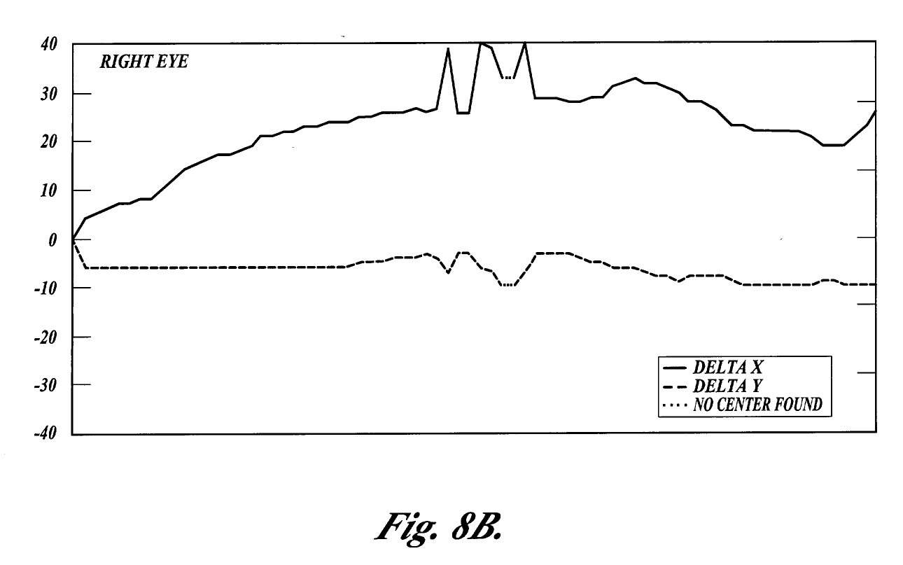

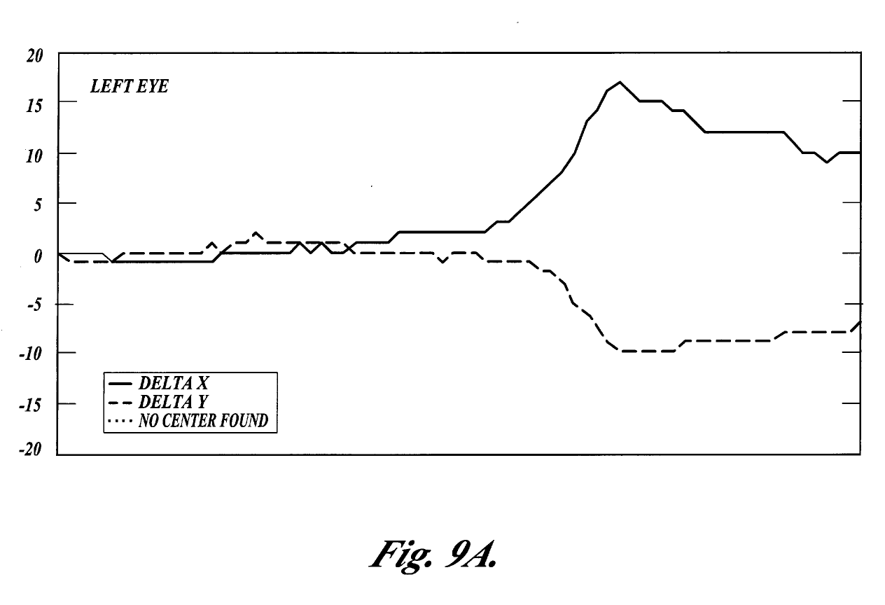

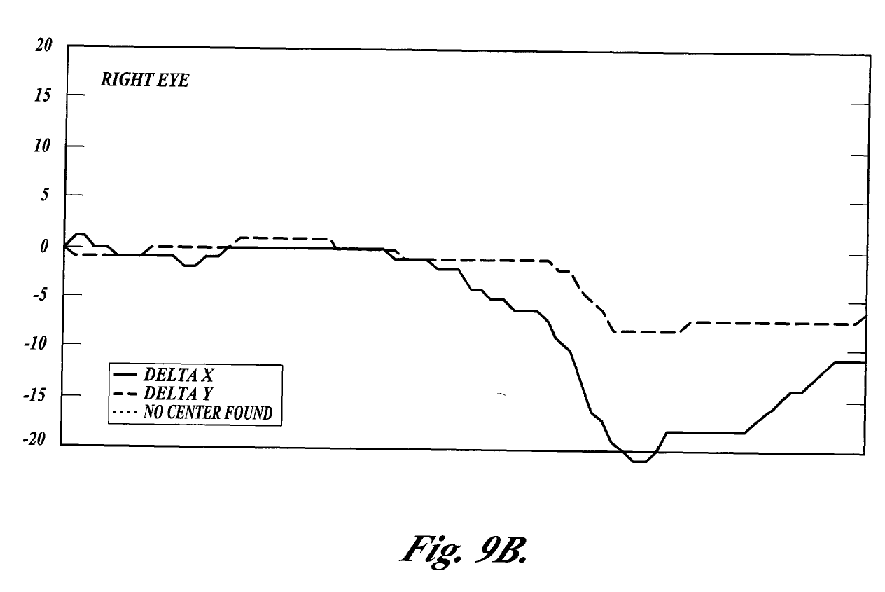

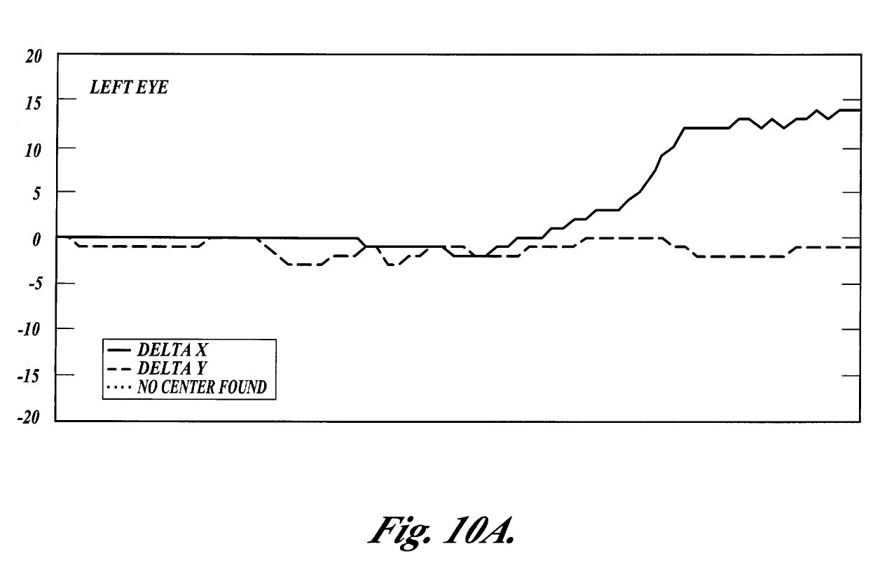

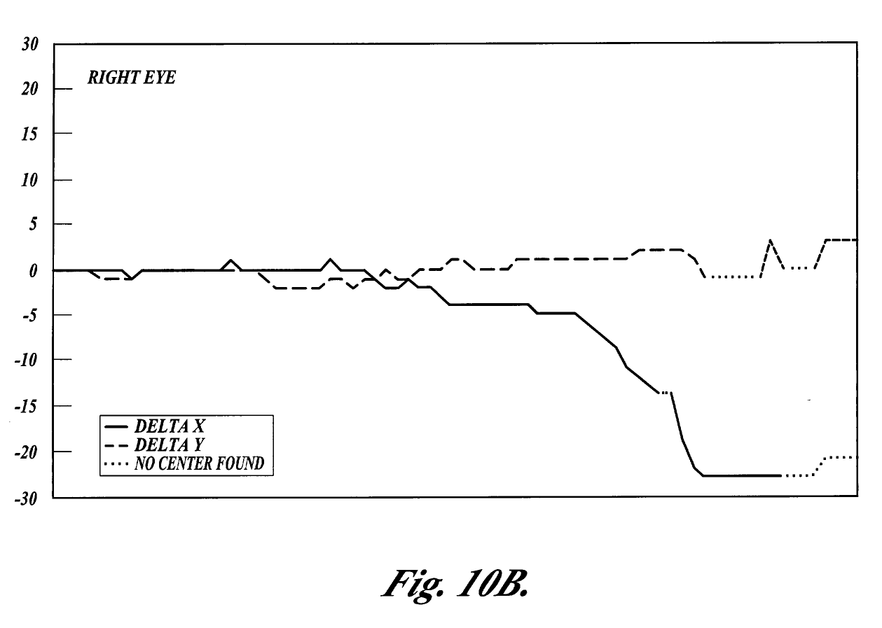

FIGS. 7A, 7B, 8A, 8B, 9A, 9B, 10A, and 10B show the progress of a patient during a treatment regime. FIGS. 7A and 7B show a plot taken before treatment. The patient cannot fully control the focus of the eyes. The right eye dropped down sharply at one point. In addition, at the similar location, the left eye loses focus for most of the time after that point. FIGS. 8A and 8B show a plot taken at about one month after beginning treatment. Both eyes still show erratic movement. However, the patient can better focus the eyes. FIGS. 9A and 9B show a plot taken three months after the beginning of treatment. At this time, the patient has even better control of both eyes. In this regard, both eyes track the moving target relatively closely. FIGS. 10A and 10B show a plot taken four months after the beginning of the treatment. The patient still maintains the control of both eyes.

### Software 200

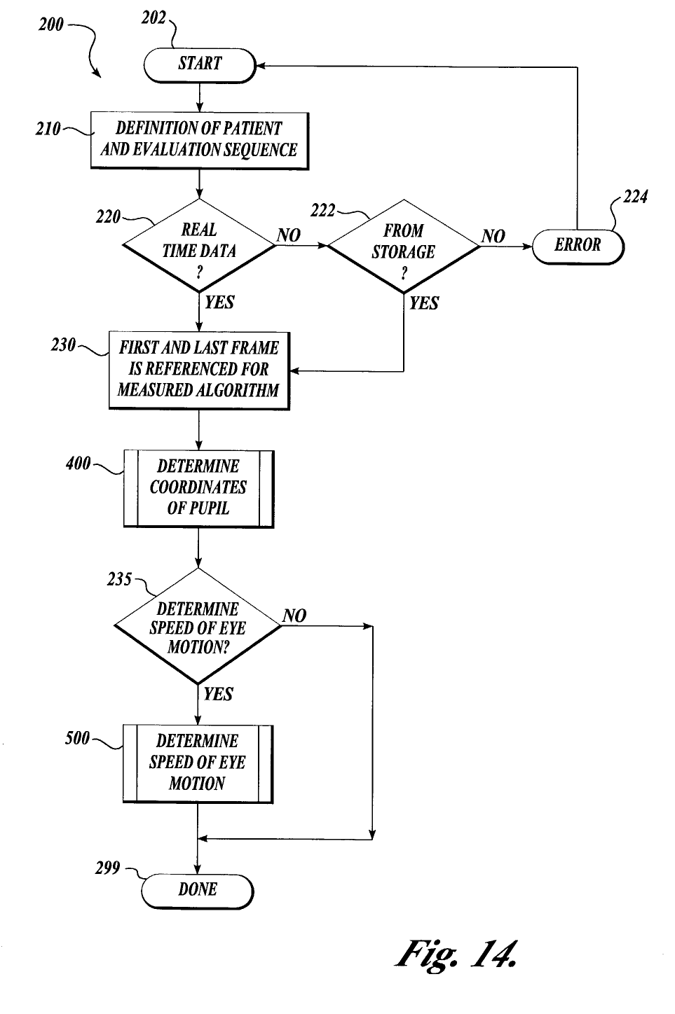

A software program may help a physician in performing some portions of the data analysis, particularly the repetitious and time-consuming tasks of viewing and analyzing the images of the patient's eyes, determining whether the eyes are focused or not, measuring the eye movement, and producing plots such as those depicted in FIGS. 6A-10B. The eye movement analysis module 200 depicted in FIG. 14 is one embodiment of a software program that may aid physicians in analyzing data collected by the medical device 10.

The eye movement analysis module 200 starts at block 202 and requests an input of a patient's identification information at block 210. The eye movement analysis module 200 then checks whether the data analysis is in real time at block 220. If it is in real time, the software retrieves data directly from the control box 14 as the data is acquired from the camera 40 and movement system 86 etc. If the data analysis is not in real time, the data is retrieved from data storage (in block 222), such as from the memory 135 of the computer 115, the recorder 90, a central storage disk farm, or another storage device or media. If there is an error in locating the necessary data, the program stops at block 224 and reports an error.

Once the image data and corresponding target position data are loaded, the software uses the patient's name as an identifier of the foregoing analysis, in block 230. However, it is apparent to one of ordinary skill in the art that numbers or arbitrary terms may be used to identify the results of the foregoing analysis for storage and later retrieval. The software starts the data analysis at block 400 where the software determines the coordinates of each pupil.

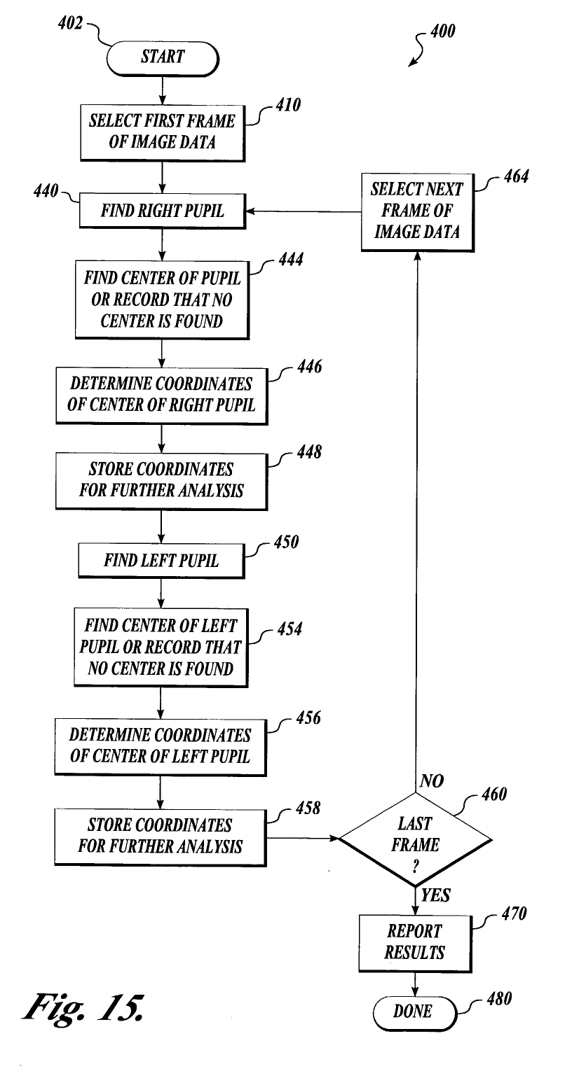

Referring to FIG. 15, the process for determining pupil coordinates are detailed. First, in block 410 a single frame of the image data is selected. Preferably, the first frame (N₁) of the image data is selected first and then the process is repeated for each successive frame of the image data until the last frame (N_{X}) is analyzed. However, if stored image data is being analyzed, the frames need not be analyzed in the order of their capture.

In block 440, the software finds the right pupil. In alternate embodiment, the software may also locate a fixed reference location of the right eye in this block. The reference location is the origin or zero location of the coordinate system used to determine the position of the pupil. The inner corner of the eye may be used as a reference location. During a manual analysis or a semi-automated analysis, the user may manually locate and mark the pupil and/or a reference location (if one is used). However, during a fully automated analysis, it is desirable to have the eye movement analysis module 200 perform this function. Because the pupil of the eye is generally dark in color, the software may automatically determine the area defined by the pupil by locating dark regions in the frame. Furthermore, automatic detection of the pupil may be aided by selective positioning of the eyes within the image. For example, the pupil may be positioned vertically near the center of the frame and horizontally on the side of the frame corresponding to the eye being analyzed. In this manner, the software need only locate a dark region within the general region where the pupil is expected. The software can locate dark regions by examining the color values of each pixel in the frame. The software may have a predetermined list of color values that are consistent with the color values found in the pupil of the eye. The software may then select from among the regions satisfying the color requirements for a region in an expected location or of a sufficient size to be the pupil of the eye. If the software is unable to locate the pupil, in the next block 444, the software will record it was unable to determine the center of the pupil.

The location of the inner corner of the eye can also be automatically determined. In one embodiment, the location of the inner corner of the eye is calculated as a function of the angle formed between the pupils as they turn toward one another. Further, a line drawn between the pupils as they are turned toward one another may also form the zero line in the Y direction. Alternatively, the location of the inner corner of the eye could be located using the eye image data. Particularly, the location of the reference point could be calculated as a function of the motion of the eye and the travel path of the target.

Next, the program proceeds to find the center of the pupil (in block 444) or record that no center could be found, and determine the coordinates of the center of the pupil (in block 446). All of the pixels in a 2-D image have a coordinate value (with an X and Y component) that represents their location within the image measured in pixels. The center of the pupil may be located by any method known in the art for determining the center of an area. One non-limiting method of locating the center of an area includes determining the location of four points the points along the area defining the pupil. These points include points that have the greatest and least vertical values and the points with the greatest and least horizontal values. If a first line is drawn between the two points with the greatest and least vertical values and a second line is drawn between the two points with the greatest and least horizontal values, the two lines will intersect in the center of the area defined by the pupil. Mathematically, the formulas for both the first and second line are determined using the formula of a straight line (y=xm+b; where m is the slope of the line) and using these formulas the intersection of the two lines may be calculated.

It is important to mention, that all of the above calculations used the X and Y coordinates of the individual pixels within the image. For the purposes of data collection, it is important to determine the coordinates of the pupils relative to a constant location. In this manner, successive measurements can be compared without the need to locate the eyes within the exact location within the image data used in previous dynamic runs. For this reason, the coordinates of the center of pupil may be calculated relative to the reference point (discussed above) or another reference point within the captured image. The reference point is the origin of an orthogonal or Cartesian coordinate system, i.e., the coordinates of the reference point may be written as (0,0). A single pixel may represent a unit on the coordinate system. Alternatively, lower resolution measuring may be used such that any number of pixels is a unit on the coordinate system. The X-coordinate of the location of the center of the pupil is calculated by subtracting the X-component of the reference location relative to the image from the X-component of the center of the pupil relative to the image. The Y-coordinate of the location of the center of the pupil is calculated by subtracting the Y-component of the reference location relative to the image from the Y-component of the center of the pupil relative to the image.

Once the coordinates of the center of the pupil are determined, the software stores the coordinates in block 448 and proceeds to analyze the left eye in block 450. The coordinates may be stored in any manner known in the art including storage in computer files and databases. Particularly, a database external to the program modules may be used to store the coordinate data. The software goes through the same process as in blocks 454, 456, and 458 as carried out in blocks 444, 446, and 448. Once both eyes corresponding to a particular target position (i.e., captured within a single frame) are analyzed, the software checks to verify that all of the frames within the image data have been analyzed in block 460. If not, the software analyzes the next frame and so on, until the last frame is completed. In block 470, the results may be reported. The report may include the pupil coordinates and corresponding target positions plotted or graphed and displayed on the computer monitor 32 (shown in FIG. 1) or printed on hardcopies by a printer/plotter, such as those shown in FIGS. 6A-10B. The software then stops in block 480.

Returning to FIG. 14, after the coordinates of the focused pupils are determined in block 400, the user can decide in decision block 235 whether to determine the speed of eye motion. If the user decides to determine the speed of eye motion, the software proceeds to block 500. The speed of eye motion may be useful when evaluating the sensory-motor response of the ocular-visual system. Otherwise, the program ends at block 299.

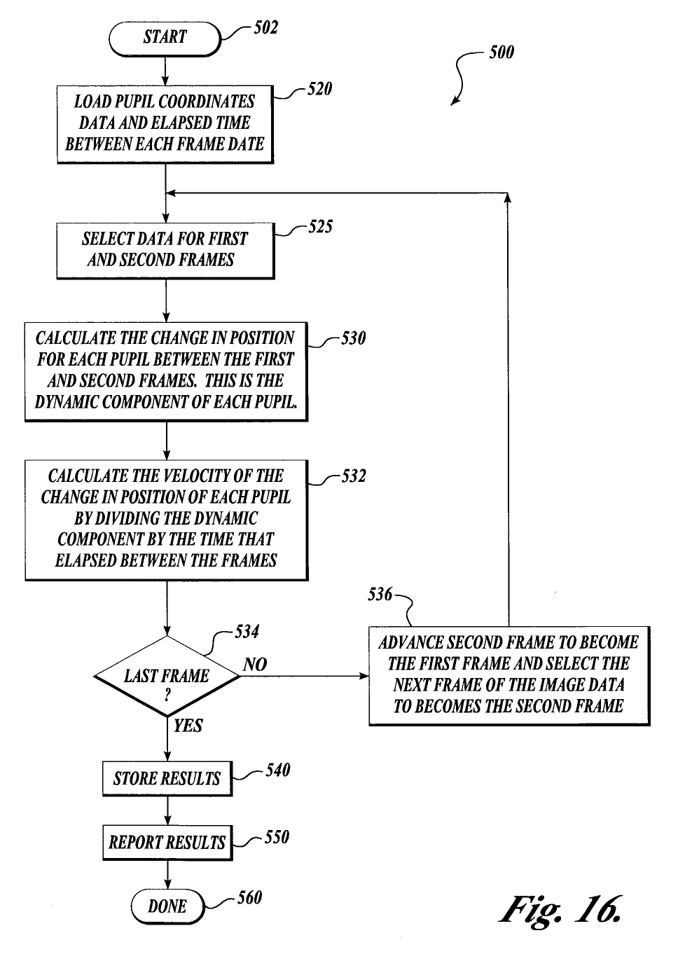

The method of determining the speed of eye motion 500 is detailed in FIG. 16. In the block 500, the program of the present invention calculates the speed of the eye motion in response to the stimulus of the target 20. The second process starts at block 502. Speed or velocity is calculated by dividing a change in position by the time required to effect that change in position. In block 520, the software loads the pupil coordinate data calculated by process 400 and the elapsed time between each frame data (recorded as discussed above). Next, in block 525 the software selects the data for the adjacent first and second frames of image data. In block 530, the dynamic component is calculated for each pupil by calculating the change of position (δd₁₂) of each pupil from the first (dn₁) to the second (dn₂) frame. Generally, the distance between any two points can be calculated using the following formula:

$$Distance = \sqrt{{( {X}_{2} - {X}_{1} )}^{2} + {( {Y}_{2} - {Y}_{1} )}^{2}}$$

Where

X₁=the x component of the coordinate of the pupil in the first frame

X₂=the x component of the coordinate of the pupil in the second frame

Y₁=the y component of the coordinate of the pupil in the first frame

Y₂=the y component of the coordinate of the pupil in the second frame

The velocity or speed expressed in linear speed or angular speed for each pupil is calculated in block 530 by dividing the change in position of each pupil (δd₁₂) by the time lapsed between the adjacent frames (t). The software then checks whether all frames of the image data have been analyzed in block 534. If not, then the software then advances the second frame so that it becomes the first frame and selects the next frame of image data to become the second frame. The software then returns to block 530. In this manner, the next two adjacent frames of the image data are analyzed next. If all frames of the image data have been analyzed, the software moves to block 540 to store calculated speed with the corresponding target positions. The software generates a report in block 550 and ends in block 560.

The software program makes it possible to perform data analysis in the real time during the examination of the patient. The physician may adjust the later portion of an examination routine according to the results of the earlier portion of the examination routine. For example, the physician may take more or fewer images, move the target faster or slower, or move the target closer or farther away from patient's eyes, depending on the reaction from the patient. The physician may also fine-tune each run, varying the speed of the target and frequency of taking images of the eyes, changing the trajectory of the target, etc. At a certain distance where the patient has trouble focusing the eyes, images may be taken at a greater frequency. The physician may use various methods to amplify or focus on the manifestation of a patient's problems, or confirm or reject a preliminary diagnosis during the examination. In this manner, the physician can obtain more information about the patient's ailments and the effect of treatment therefor, so that the physician need perform fewer diagnosis or monitoring sessions with a patient.

The present invention can help physicians in many ways. For example:

1. To identify the source of the patient's physical, and especially neurological, problems. A preliminary assessment can occur to identify and determine the source of the patient's problems and sensory-motor condition. It is important to determine whether the condition results from damage (whether within or outside of the central nervous system) or dysfunction of the central nervous system. Dysfunction can be corrected, while damage typically cannot. Structural damage outside the central nervous system is not considered damage for the purposes of this invention.

2. To predict expected benefits of various management/treatment regimes. An initial assessment also allows physicians or caregivers to foresee/predict the likely outcome of a restoration program. Problems resulting solely from dysfunction may be successfully treated. Even where there is both dysfunction and damage to the central nervous system, there can be functional gain in many cases.

3. To monitor the patient's recovery and functional gains. Recovery of normal function that is stable and lasting is of the greatest value. For physicians, predicting the “likely outcome” (as mentioned above) means predicting long-term benefit. A restoration program initiates a functional healing momentum, which should continue after a treatment program has ended.

4. To optimize gain of sensory-motor functions. Dysfunction does not necessarily mean illness. The restoration program objective is to optimize function and skill levels in the physical and cognitive areas. By optimizing function and skill levels, the methods and protocols lead to optimal performance in a variety of contexts, including: professional, educational, and athletics.

5. To provide the desired precise measurement concerning the Fine Postural System.

While specific and preferred embodiments of the invention have been illustrated and described, it will be appreciated that various changes can be made therein without departing from the spirit and scope of the invention.

The embodiments of the invention in which an exclusive property or privilege is claimed are defined as follows:

1. A medical device for capturing eye movement in response to a moving target, comprising:
a. a camera for capturing images of at least one eye focused on at least one eye; and
b. a movement system for supporting a target and moving the target relative to at least one eye;
c. wherein the camera captures images of at least one eye as the movement system moves the target relative to at least one eye.

2. The medical device of claim 1, wherein the movement system is coupled to a movement system controller for controlling the movement of the movement system.

3. The medical device of claim 2, wherein the movement system controller periodically records the position of the target as the movement system moves the target.

4. The medical device of claim 2, wherein the movement system controller comprises a computing device.

5. The medical device of claim 4, wherein the computing device comprises a computer readable medium having computer executable components for controlling the movement system.

6. The medical device of claim 1, wherein the movement system comprises at least one internal sensor operationally coupled to the movement system for determining the position of the target.

7. The medical device of claim 6, wherein at least one internal sensor is coupled to a recorder that periodically records the position of the as the movement system moves the target.

8. The medical device of claim 1, wherein the movement system moves the target along a linear path between a first predetermined location spaced from the eye and a second predetermined location nearer the eye.

9. The medical device of claim 1, wherein the camera is coupled to a camera controller for controlling the capture of images of at least on eye.

10. The medical device of claim 9, wherein the camera controller comprises a computing device.

11. The medical device of claim 10, wherein the computing device comprises a computer readable medium having computer executable components for controlling the camera.

12. The medical device of claim 1, wherein the camera is coupled to a recorder for recording the images captured by the camera.

13. The medical device of claim 12, wherein the recorder comprises a computing device recording the images on a computer readable medium.

14. The medical device of claim 12, wherein the recorder comprising an electronic digital recorder storing information in digital format on a computer readable medium.

15. The medical device of claim 1, wherein the camera is coupled to a communication interface for communicating captured images to a computing device.

16. The medical device of claim 15, wherein the computing device receives images from the camera as the images are captured.

17. The medical device of claim 1, wherein the camera captures images of both eyes.

18. The medical device of claim 1, further comprising a headrest extending forwardly from the camera.

19. The medical device of claim 1, further comprising an eye illuminator assembly positioned relative to at least one eye to provide illumination to at least one eye.

20. The medical device of claim 19, wherein the eye illuminator assembly comprises a conduit extending toward at least one eye and supporting an eye illuminator light providing illumination to at least one eye.

21. The medical device of claim 20, wherein the eye illuminator assembly further comprising a light source at its proximal end that transmits light to the distal end of the conduit to illuminate at least one eye.

22. The medical device of claim 21, wherein the eye illuminator assembly comprises a fiber optic bundle within the flexible conduit to transmit light from the light source to the distal end of the flexible conduit for illuminating at least one eye.

23. The medical device of claim 1, wherein the movement system comprising:
a. a support rod supporting the target at the distal end of the support rod; and
b. a drive mechanism motor for supporting, extending, and retracting the support rod relative to at least one eye.

24. The medical device of claim 23, wherein the drive mechanism further comprises:
a. idler wheels and a drive wheel engaged with and supporting the support rod;
b. a motor drivingly connected to the drive wheel; and
c. a sensor operationally connected to at least one of the group including the drive wheel, motor, idler wheels, and support rod generating a signal indicating the position of the support rod relative to the control box.

25. The medical device of claim 24, wherein the target is lighted.

26. The medical device of claim 1, wherein the camera comprises an electronic digital camera that transmits images in an electronic digital format.

27. The medical device of claim 1, wherein the control box is attached to one end of an elongate support arm, and the other end of the support arm being mountable to a fixed location at the location where the medical device is to be used.

28. A system for capturing and analyzing eye movement in response to a moving target, comprising:
a camera;
a movement system;
a target;
a computing device comprising
a. a processing unit;
b. a camera communication interface for communicating with the camera wherein the camera communication interface is coupled to both the processing unit and the camera so that a plurality of images captured by the camera are transmitted to and received by the processing unit;
c. a movement system communication interface for communicating with the movement system coupled to both the processing unit and the movement system enabling the processing unit to send signals to the movement system; and
d. a storage medium coupled to the processing unit, the storage medium storing program code implemented by the processing unit for
1. controlling the movement system by sending signals to the movement system through the movement system communication interface and determining the position of the target coupled to the movement system at the time each image of the plurality is captured; and
2. analyzing the movement of at least one eye in response to the moving target by analyzing the plurality of images captured by the camera;
wherein the camera is focused on at least one eye and captures images while the movement system is moving the target; and the movement system supports and moves the target positioned in front of at least one eye in response to signals sent by the processing unit.

29. The system of claim 28, wherein the position of the target attached to the movement system is determined as a function of the signals sent to the movement system that control the movement of the movement system.

30. The system of claim 28, further comprising at least one sensor coupled to the movement system for determining the position of the movement system.

31. The system of claim 30, further comprising a position communication interface coupled to at least one sensor and the processing unit for communicating the position of the movement system to the processing unit.

32. The system of claim 28, wherein the position of the target attached to the movement system is determined as a function of signals sent from the movement system to the processing unit.

33. The system of claim 28, wherein the storage medium further stores program code implemented by the processing unit for recording the position of the target.

34. The system of claim 28, wherein analyzing the plurality of images captured by the camera comprises:
a. determining a coordinate system wherein an origin of the coordinate system appears within each image of the plurality of images;
b. selecting each image from the plurality of images one at a time;
c. determining the location of the pupil of at least one eye in the selected image;
d. determining the center of the pupil of at least one eye in the selected image; and
e. determining the coordinates of the center of the pupil within the coordinate system relative to the origin;

35. The system of claim 28, wherein analyzing the plurality of images captured by the camera comprises plotting the coordinates of the center of the pupil for each image of the plurality of images.

36. The system of claim 28, wherein analyzing the plurality of images captured by the camera comprises plotting the coordinates of the center of the pupil for each image of the plurality of images against the position of the target when each image was captured.

37. A method for assisting in diagnosing, treating, and monitoring a patient with a malfunction of the central nervous system, comprising:
a. positioning a target in front of at least one eye of the patient;
b. positioning a camera to capture a series of images of at least one eye of the patient;
c. moving the target along a trajectory relative to at least one eye of the patient after the patient has focused at least one eye on the target;
d. capturing the series of images of at least one eye with the camera as the target moves;
e. determining the position of the target at the time each image is captured; and
f. analyzing the position of a pupil of at least one eye in each image of the series of images captured by the camera.

38. The method of claim 37, further comprising selecting the trajectory for the target to follow while in motion.

39. The method of claim 37, wherein the trajectory is linear.

40. The method of claim 37, wherein the target is positioned between the two eyes of the patient.

41. The method of claim 37, wherein the trajectory has start and end location and the start locations is farther from the patient than the end location.

42. The method of claim 37, wherein the trajectory comprises a start location that is the location of the target at the start of movement along the trajectory and moving the target along the trajectory comprises a start time at which the target begins to move and a speed at which the target moves along the trajectory and the position of the target at the time each image is captured is determined as a function of the trajectory, start location, speed, and start time.

43. The method of claim 37, wherein the position of the target at the time each image is captured is determined by a movement system that controls the movement of the target.

44. The method of claim 42, wherein the position of the target at the time each image is captured is recorded by a movement system that controls the movement of the target.

45. The method of claim 37, further comprising recording the position of the target at approximately the time each image is captured.

46. The method of claim 37, wherein analyzing the position of the pupil of at least one eye in each image of the series of images captured by the camera comprises:
f. determining a coordinate system wherein an origin of the coordinate system appears within each image of the series of images;
g. serially selecting each image from the series of images;
h. determining the location of the pupil of at least one eye in the selected image;
i. determining the center of the pupil of at least one eye in the selected image; and
j. determining the coordinates of the center of the pupil within the coordinate system relative to the origin;

47. The method of claim 46, wherein the coordinate system comprises an orthogonal X-axis and Y-axis.

48. The method of claim 47, wherein the coordinates of the center of the pupil comprise an X-value along the X-axis and Y-value along the Y-axis.

49. The method of claim 48, wherein analyzing the position of the pupil of at least one eye in each image of the series of images captured by the camera comprises plotting the X-value of the coordinates of the center of the pupil in each image within the series of images against the position of the target when the image was captured.

50. The method of claim 48, wherein analyzing the position of the pupil of at least one eye in each image of the series of images captured by the camera comprises plotting the Y-value of the coordinates of the center of the pupil in each image within the series of images against the position of the target when the image was captured.

51. The method of claim 47, wherein the coordinate system further comprises a Z-axis orthogonal to both the X-axis and Y-axis and passing through the origin.

52. The method of claim 51, wherein determining the position of the target at the time each image is captured comprises determining the coordinates of the target within the coordinate system relative to the origin so that the position of the target comprises a Z-value along the Z-axis on the coordinate system.

53. The method of claim 52, wherein analyzing the position of the pupil of at least one eye in each image of the series of images captured by the camera comprises plotting the X-value of the coordinates of the center of the pupil in each image within the series of images against the Z-value of the coordinates of the target when the image was captured.

54. The method of claim 52, wherein analyzing the position of the pupil of at least one eye in each image of the series of images captured by the camera comprises plotting the Y-value of the coordinates of the center of the pupil in each image within the series of images against the Z-value of the coordinates of the target when the image was captured.

55. The method of claim 46, further comprising recording the coordinates of the center of the pupil for each image of the series of images onto a readable medium.

56. The method of claim 46, further comprising analyzing the speed of movement of the pupil of at least one eye within the series of images captured by the camera.

57. The method of claim 56, wherein analyzing the speed of movement of the pupil of at least one eye within the series of images captured by the camera comprises:
a. selecting a first set of coordinates for the center the pupil of one eye within a first image;
b. selecting a second set of coordinates for the center of the same pupil within a successive image of the series of images;
c. determining a linear distance between the first set of coordinates and the second set of coordinates;
d. determining a time interval that elapsed between capturing the first and second images; and
e. calculating the speed of movement of the pupil of the eye as a function of the linear distance between the first set of coordinates and the second set of coordinates and the time interval that elapsed between capturing the first and second images.

58. The method of claim 57, wherein camera captures images at a predetermined capture rate and the time interval that elapsed between capturing the first and second images is determined as function of the capture rate.

59. A computer readable medium having computer executable components for analyzing eye motion in response to an external visual stimulus, the computer executable components comprising an eye movement analysis module for
a. identifying a pupil in each image of a series of images of at least one eye of a patient captured while observing the external visual stimulus;
b. determining a coordinate system having an origin common to all images in the series of images;
c. determining the coordinates of the center of the pupil within the coordinate system relative to the origin for each image of a series of images; and
d. calculating the distance of the external visual stimulus from the pupil for each image of a series of images.

60. The computer readable medium of claim 59, wherein the distance of the external visual stimulus from the pupil is calculated as a function of the coordinates of the external visual stimulus within the coordinate system relative to the origin.

61. The computer readable medium of claim 59, further comprising a movement system module for determining the position of the external visual stimulus relative to at least one eye.

62. The computer readable medium of claim 61, wherein the movement system module comprises computer executable components for communicating position information concerning the external visual stimulus to the eye movement analysis module and the eye movement analysis module calculates the distance of the external visual stimulus from the pupil for each image in the series of images as a function of position data information provided by the movement system module.

63. The computer readable medium of claim 61, wherein the movement system module determines the position of the external visual stimulus relative to at least one eye by having computer executable components for reading position data recorded on a computer readable medium.

64. The computer readable medium of claim 61, wherein the movement system module records the position of the external visual stimulus relative to at least one eye by having computer executable components for receiving position data from a movement system and computer executable components for recording the position data.

65. The computer readable medium of claim 61, wherein the movement system module comprises computer executable components for recording the position of the external visual stimulus relative to at least one eye.

66. The computer readable medium of claim 65, wherein the eye movement analysis module calculates the distance of the external visual stimulus from the pupil for each image in the series of images as a function of position data recorded by the movement system module.

67. The computer readable medium of claim 61, wherein the movement system module comprises computer executable components for recording the position of the external visual stimulus relative to at least one eye at the time each image captured.

68. The computer readable medium of claim 59 further comprising a camera module for controlling a camera used to capture the series of images.

69. The computer readable medium of claim 68, wherein the camera module comprises computer executable components for directing the camera to capture images.

70. The computer readable medium of claim 68, wherein the camera module comprises computer executable components for determining a capture rate at which the camera captures the images in the series of images.

71. The computer readable medium of claim 59 further comprising a recording module for recording the series of images captured by the camera.

72. The computer readable medium of claim 59, wherein the external visual stimulus traverses along a path of a predetermined length at a predetermined speed and the external visual stimulus begins to move along the path at a start time and the eye movement analysis module calculates the distance of the external visual stimulus from the pupil for each image in the series of images as a function of the predetermined length of the path, the speed, and the start time.

73. The computer readable medium of claim 59, wherein the eye movement analysis module comprises computer executable components for providing a report of the coordinates of the center of the pupil and the distance of the external visual stimulus from the pupil for each image of a series of images.

74. The computer readable medium of claim 59, wherein the eye movement analysis module comprises computer executable components for displaying the coordinates of the center of the pupil and the distance of the external visual stimulus from the pupil for each image of a series of images.
